(19) United States
(12) Patent Application Publication
(10) Pub. No.: US 2006/0052717 A1
(43) Pub. Date: Mar. 9, 2006

(54) Method and system for evaluating cardiac ischemia

(76) Inventors: Dale H. Mugler, Hudson, OH (US); Soumyadipta Acharya, Baltimore, MD (US); Raghavan Gopalakrishnan, Parma, OH (US)

Correspondence Address: ROETZEL AND ANDRESS, 222 SOUTH MAIN STREET, AKRON, OH, 44308, US

(21) Appl. No.: 11/216,235

(22) Filed: Aug. 31, 2005

Related U.S. Application Data
(60) Provisional application No. 60/605,951, filed on Aug. 31, 2004.

(51) Int. Cl. A61B 5/0402

(52) U.S. Cl. 600/509

(57) ABSTRACT

The present invention relates to methods and systems for evaluating abnormalities in electrocardiograms (ECGs), including abnormalities associates with cardiac ischemia. More particularly, the present invention relates to an automated system and method for interpreting any abnormalities present in an electrocardiogram (ECG), including those abnormalities associated with cardiac ischemia. In one embodiment, the present invention relates to a method for monitoring abnormalities in an ECG, the method comprising the steps of: (a) gathering at least one ECG; (b) subjecting the at least one ECG to a QRS detection algorithm in order to scan for R-peak location; (c) calculating the Hermite coefficients corresponding to the individual ECG complexes from each individual ECG; and (d) subjecting the Hermite coefficients to a Neural Network in order to determine the present and/or absence of ECG abnormalities.

### RELATED APPLICATION DATA

This application claims priority to previously filed U.S. Provisional Application No. 60/605,951 filed on Aug. 31, 2004, entitled “Real Time Monitoring of Ischemic Changes in Electrocardiograms”, and is hereby incorporated by reference in its entirety.

### FIELD OF THE INVENTION

The present invention relates to methods and systems for evaluating abnormalities in electrocardiograms (ECGs), including abnormalities associates with cardiac ischemia. More particularly, the present invention relates to an automated system and method for interpreting any abnormalities present in an electrocardiogram (ECG), including those abnormalities associated with cardiac ischemia.

### BACKGROUND OF THE INVENTION

Heart attacks and other ischemic events of the heart are among the leading causes of death and disability in the United States. In general, the susceptibility of a particular patient to heart attack or the like can be assessed by examining the heart for evidence of ischemia (insufficient blood flow to the heart tissue itself resulting in an insufficient oxygen supply) during periods of elevated heart activity. Of course, it is highly desirable that the measuring technique be sufficiently benign to be carried out without undue stress to the heart (the condition of which might not yet be known) and without undue discomfort to the patient.

The cardiovascular system responds to changes in physiological stress by adjusting the heart rate, which adjustments can be evaluated by measuring the surface ECG R—R intervals. The time intervals between consecutive R waves indicate the intervals between the consecutive heartbeats (RR intervals). This adjustment normally occurs along with corresponding changes in the duration of the ECG QT intervals, which characterize the duration of electrical excitation of cardiac muscle and represent the action potential duration averaged over a certain volume of cardiac muscle. Generally speaking, an average action potential duration measured as the QT interval at each ECG lead may be considered as an indicator of cardiac systolic activity varying in time.

Recent advances in computer technology have led to improvements in automatic analyzing of heart rate and QT interval variability. It is known that the QT interval's variability (dispersion) observations performed separately or in combination with heart rate (or RR-interval) variability analysis provides an effective tool for the assessment of individual susceptibility to cardiac arrhythmias.

As is noted above, ischemic heart disease is a common cause of death and disability in industrialized countries. The ECG is one of the most important tools for the diagnosis of ischemia. Long term continuous ECG monitoring is found to offer more prognostic information than the standard 12 lead ECG, concerning ischemia. Given the usefulness of ECG in identifying ischemia, there is a need in the art for a reliable computer based method to interpret ECG results in order to identify the abnormalities associated with not only ischemia, but other types of heart disease as well.

### SUMMARY OF THE INVENTION

The present invention relates to methods and systems for evaluating abnormalities in electrocardiograms (ECGs), including abnormalities associates with cardiac ischemia. More particularly, the present invention relates to an automated system and method for interpreting any abnormalities present in an electrocardiogram (ECG), including those abnormalities associated with cardiac ischemia.

In one embodiment, the present invention relates to a method for monitoring/detecting abnormalities in an ECG, the method comprising the steps of: (a) gathering at least one ECG; (b) subjecting the at least one ECG to a QRS detection algorithm in order to scan for R-peak location; (c) calculating the Hermite coefficients corresponding to the individual ECG complexes from each individual ECG; and (d) subjecting the Hermite coefficients to a Neural Network in order to determine the present and/or absence of ECG abnormalities.

In another embodiment, the present invention relates to a computer system designed to carry out a method for monitoring/detecting abnormalities in a ECG, the computer system comprising: at least one power source; at least one input device; at least one display; and at least one memory device, wherein the computer system is designed to act as a Neural Network.

In still anther embodiment, the present invention relates to a a method for monitoring abnormalities in an ECG, the method comprising the steps of: (a) gathering at least one ECG; (b) subjecting the at least one ECG to a QRS detection algorithm in order to scan for R-peak location; (c) calculating the Hermite coefficients corresponding to the individual ECG complexes from each individual ECG; and (d) subjecting the Hermite coefficients to a Neural Network in order to determine the present and/or absence of ECG abnormalities, wherein the ECG abnormalities being monitored/detected are associated with cardiac ischemia, and wherein Steps (b) and (c) are conducted simultaneously.

### BRIEF DESCRIPTION OF THE DRAWINGS

FIG. 1 illustrates the first six Hermite functions where the functions have a dilation parameter of 3;

FIG. 2 illustrates dilated discrete Hermite functions u_{3,b} for k=3, and three different dilation values b=1.25, 1.5 and 2.0;

FIG. 3 illustrates the first six Hermite functions u_{k,b} for k=0, 1, . . . , 5 for the b=1 undilated case, n=128;

FIG. 4 illustrates the first six dilated discrete Hermite functions u_{k,b} for k=0, 1, . . . 5 for the b=4 dilated case, n=128,

FIG. 5 illustrates an approximation of sinusoid by an expansion with just two discrete Hermite functions;

FIG. 6 is an illustration of an ECG signal approximation with six Hermite functions, b≈1.43;

FIG. 7 is an illustration of the ECG signal of FIG. 6 approximated using 12 Hermite functions and a larger scale parameter b=2.06;

FIG. 8 is an illustration of an ECG signal approximation using 12 Hermite functions, b=2.60;

FIG. 9 is an illustration of the ECG signal of FIG. 8 approximated using six Hermite functions and a smaller dilation parameter b≈1.36;

FIG. 10 is an illustration of an ECG signal approximation using six Hermite functions, b≈1.51;

FIG. 11 is an illustration of a centered Fourier transform ECG signal from FIG. 9 using six Hermite functions;

FIG. 12(a) is an original electrocardiogram;

FIG. 12(b) is a reconstruction of the electrocardiogram of FIG. 12(a) using 50 Hermite functions;

FIG. 13 illustrates the adaptation of second Hermite function to the shape of an electrocardiogram (ECG) (to enable visualization of the Hermite expansion, a small offset is present in FIG. 13);

FIG. 14 is a block diagram of a system, according to one embodiment of the present invention, that is designed to monitor for ischemia in long term electrocardiogram signals;

FIG. 15(a) is a illustration of various segments of ECG signals from record e011 of the European ST-T database and the corresponding Neural Network output generated by the ischemia monitoring system/method of the present invention; and

FIG. 15(b) is a illustration of various segments of ECG signals from record e0603 of the European ST-T database and the corresponding Neural Network output generated by the ischemia monitoring system/method of the present invention.

### DETAILED DESCRIPTION OF THE INVENTION

The present invention relates to methods and systems for evaluating abnormalities in electrocardiograms (ECGs), including abnormalities associates with cardiac ischemia. More particularly, the present invention relates to an automated system and method for interpreting any abnormalities present in an electrocardiogram (ECG), including those abnormalities associated with cardiac ischemia.

In one embodiment, the present invention utilizes Hermite functions to evaluating abnormalities in electrocardiograms (ECGs), including abnormalities associates with cardiac ischemia. The Hermite functions utilized by the present invention are generated as explained below. Although, it should be noted that the present invention is not limited to just the Hermite functions and/or the method detailed below that is used to generate Hermite functions.

Generation of Hermite Functions

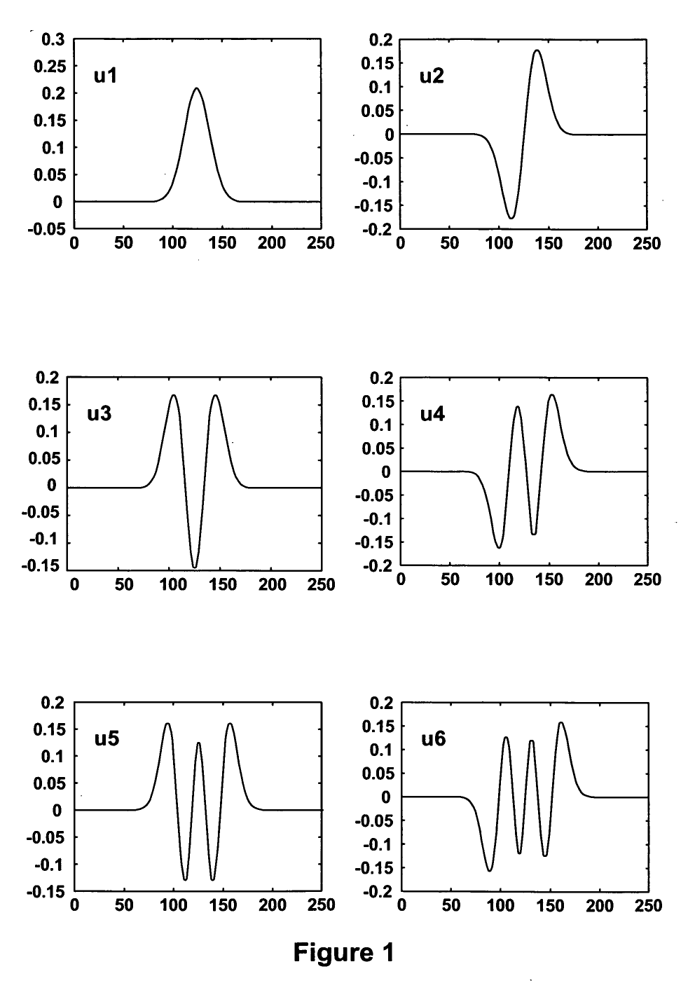

The dilated discrete Hermite functions are eigenvectors of a symmetric tridiagonal matrix T_{b} that commutes with the centered Fourier matrix. By specifying its main diagonal and one of the off-diagonals, a complete description of the tridiagonal matrix can be done. The main diagonal of T_{b} is the vector shown below in Equation (1).
−2 cos(πnτ)sin(πμτ)sin(π(n−μ−1)τ) (1)
The off diagonal vector for T_{b} is given by Equation (2), as shown below:
sin(πnτ)sin(π(n−μ)τ) (2)
for 0≦μ≦n−1 and τ=1/(nb²). The effect of the dilation parameter b is that the function broadens as its value increases. The advantages of this technique are that fast algorithms exist to compute the set of eigenvectors of a tridiagonal matrix and this set of eigenvectors are orthonormal, hence the coefficients contain independent information. FIG. 1 shows the first six Hermite functions with a dilation parameter of 3 (b=3).
Construction of the Dilated Discrete Hermite Functions

The dilated discrete Hermite functions utilized in one embodiment of the present invention are eigenvectors of a symmetric tridiagonal matrix T_{b} that will be described herein. Because of this construction, these vectors form an orthonormal basis for n dimensions, where n is the length of the signal one wishes to model. This tridiagonal matrix commutes with the centered Fourier matrix defined in Equation (3).
F_{C,b}[i,k]=e(2πj/n)(i−a)(k−a)/b² (3)
Because of that, although the eigenvalues are different, the eigenvectors of T_{b} are also eigenvectors of the centered Fourier matrix F_{C,b}.

A tridiagonal matrix that commutes with the Fourier matrix was originally discovered by Grünbaum (see “The Eigenvectors of the Discrete Fourier Transform: A Version of the Hermite Functions,” J. Math. Anal. Appl., Vol. 88, No. 2, pp. 355-363, 1982). Grünbaum gave two forms of it, one for the Fourier matrix shifted half-way around from the traditional case (a=n/2 as in Equation (3)) and one for the traditional case (a=0). Details that show how to alter Grünbaum's definition so that it depends on the parameter a, and such that the new matrices always commute with the corresponding shifted Fourier matrices, are given in the article entitled “Shifted Fourier Matrices and their Tridiagonal Commutors,” by S. Clary et al., SIAM J. Matrix Anal. Appl.

Since the generating matrix T_{b} is symmetric and tridiagonal, a complete description of it can be done by specifying its main diagonal and: one of the off-diagonals. The main diagonal of T_{b} is the vector shown in Equation (1) above, where 0≦μ≦n−1 and τ=1/(nb²). Define the off-diagonal vector for T_{b} as the vector shown below:
sin(πμτ)sin(π(n−μ)τ)
for 1≦μ≦n−1.

The set of eigenvectors of T_{b} are the discrete dilated Hermite functions, and they may be indexed in different, equivalent ways. The index k could stand for the number of zero-crossings of the eigenvector. Alternatively, let k=0 index the eigenvector corresponding to the largest eigenvalue, k=1 for the next largest, and so on.

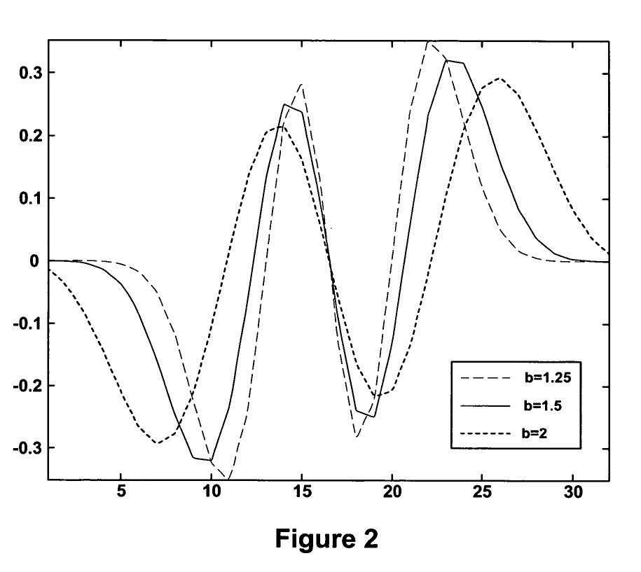

FIG. 2 shows the effect of the dilation parameter b by sketching u_{3,b} for b=1.25, 1.5 and 2.0, and k=3. With Ψ₃(t) as the continuous Hermite function with three zero-crossings, note that each is an approximation to Ψ₃(t/b) for the appropriate value of the dilation parameter. As can be seen in the FIG. 2, the Hermite function dilates further as b increases.

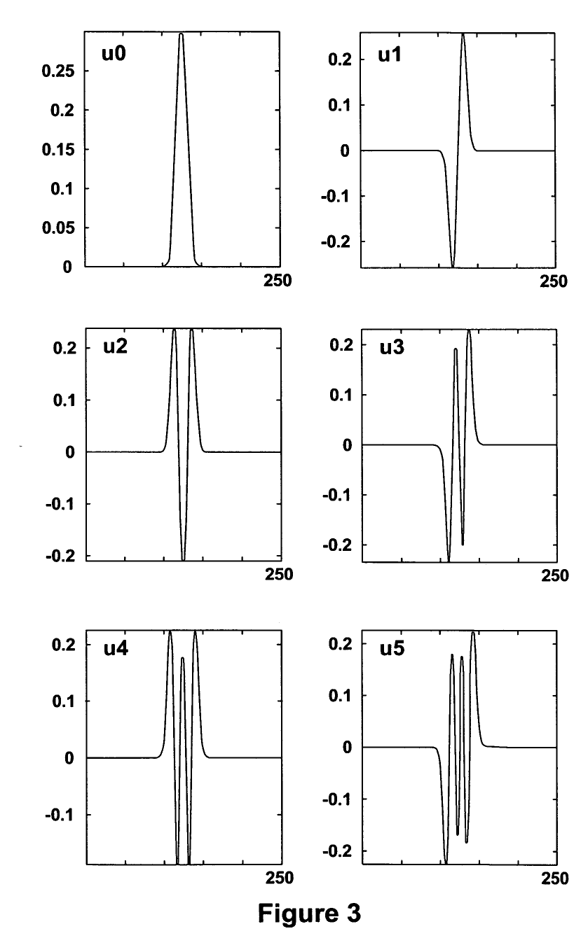

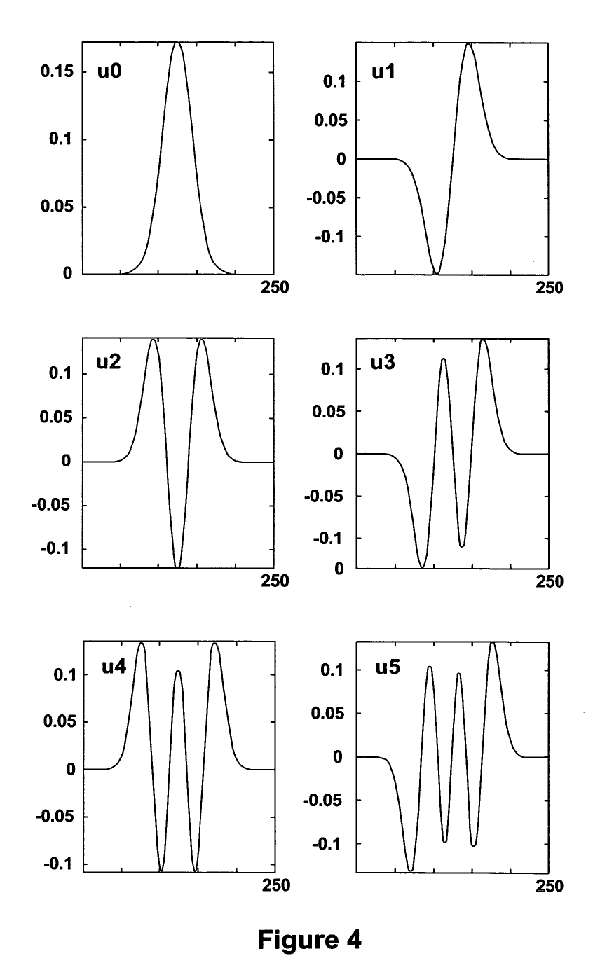

FIG. 3 shows the first six eigenvectors of T_{b} for the case when b=1. For many digital signals, only a few of the low-indexed dilated discrete Hermite functions need to be used for approximations. The effect of the scale or dilation parameter b is that the functions broaden as the value of b increases. See FIG. 4, which illustrates the first six dilated discrete Hermite functions u_{k,b} for k=0, 1, . . . , 5 for the b=4 dilated case, n=128. The value of b is important for the approximations used for applications.

Discrete Dilated Hermite Functions as Approximations to Continuous Hermite Functions

With the tridiagonal matrix Tb defined as above, next one should turn to the approximating properties of its eigenvectors. Suppose that Ψₖ(t) for k≧0 are the continuous Hermite functions, defined by Equation (4) below.

$${Ψ}_{k} ( t ) = \frac{1}{\sqrt{{2}^{k} k ! \sqrt{π}}} {H}_{k} ( t ) {ⅇ}^{- {t}^{2} / 2} \qquad ( 4 )$$

where Hₖ(t) are the Hermite polynomials. The polynomials can be calculated recursively by H₀(t)=1, H₁(t)=t and Hₖ(t)=2tHₖ₋₁(t)−2(k−1)Hₖ₋₂(t) for k≧2.

As noted above, the eigenvectors of T_{b} may be ordered based on the size of the corresponding eigenvalue. Let u_{k,b} be the kth eigenvector of T_{b}, for k=0, 1, . . . , n−1 and for dilation parameter b, ordered so that k=0 indexes the eigenvector corresponding to the largest eigenvalue, k=1 indexes the eigenvector corresponding to the next largest eigenvalue, etc. With this ordering of eigenvectors, the index of the eigenvector matches the index of the continuous Hermite function that it approximates. Assuming the u_{k,b} to be normalized, as is the standard for software packages that produce eigenvectors.

Let J_{n,b} be the set of n equally-spaced real numbers that is centered about 0 and whose adjacent points are separated by Δt/b for a dilation parameter b, with Δt=\sqrt{2π/n}. The set can simply be symbolized by J, for the special undilated case when b=1. This set of points is the set of sampling points at which the dilated discrete Hermite functions will approximate the continuous Hermite functions. This set of points is shown below in Equation (5).
J_{n,b}={mΔt/b} (5)

$$for m = - \frac{n - 1}{2} , - \frac{n - 1}{2} + 1 , … , \frac{n - 1}{2} .$$

That is, the first element of J_{n,b} is

$$- \frac{n - 1}{2} \frac{Δ t}{b}$$

and the last element is

$$\frac{n - 1}{2} \frac{Δ t}{b} .$$

The dilated discrete Hermite functions u_{k,b} approximate the continuous Hermite functions Ψₖ(t) at the points J_{n,b} from Equation (5). Symbolically, u_{k,b}[m]≈Ψₖ(tₘ), where tₘ, is the mth term in the ordered set Jₙ of (3). That is, the kth eigenvector u_{k,b} approximates the similarly-indexed classical Hermite function sampled at the set of points J_{n,b} (and normed to 1). Note that the vector on the right side of this equation must also he of unit norm to satisfy this approximation, as the eigenvectors are already normalized in this way. It should be noted that the error in the approximation increases as the index k increases. For small values of k the error is very small, and increases gradually as k increases. One of the advantages of the set of dilated discrete Hermite functions is that each set, for fixed b, is an orthonormal basis because this set consists of eigenvectors of a symmetric, tridiagonal matrix.

Discrete Hermite Expansions of Signals

Given a digital signal x of length n, the discrete Hermite expansion (Equation (6)) of x is simply an expansion of an n-dimensional digital signal in a particular orthonormal basis. This expansion has the form shown in Equation (6) below.

$$x = {\sum }_{k = 0}^{n - 1} {c}_{k , b} {u}_{k , b} \qquad ( 6 )$$

with coefficients
c_{k,b}═<x,u_{k,b>} (7)
given by standard inner products of the input signal with the discrete dilated Hermite functions.

Digital signals that are even or odd are especially easy to represent in an expansion of dilated discrete Hermite functions. Similar to the continuous Hermite functions, u_{k,b} is even or odd, depending on whether k is even or odd, k=0,1, . . . , n−1. This property is valid for any value of the dilation parameter b. For example, if input x is even, only even-indexed u_{k,b} will have non-zero coefficients. For a more general signal, it is important to emphasize that any digital signal has a representation as shown in Equation (6) since this set of discrete Hermite functions provides an orthonormal basis. There are, however, two parameters to determine in order that the expansion have as few non-zero coefficients as possible; the two parameters available for determining the expansion are (i) the center and (ii) the dilation parameter value b.

For a digital signal obtained from electrophysiological measurements, there is often a zero-crossing of the signal near the middle of the finite time support, and that would provide the center point for the expansion. The dilation parameter b is a new possibility for discrete Hermite functions, as this is the first formal announcement of dilated discrete Hermite functions. In applications to ECG signals, the center point is determined by a standard QRS detection algorithm and the value of b will he chosen so that feature points in the ECG signal match those of a similar u_{k,b} vector.

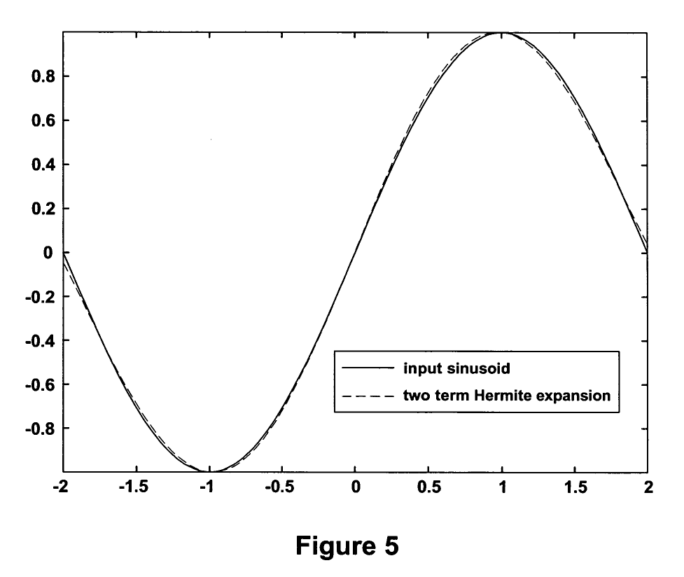

As a simple example of an Hermite expansion, consider the expansion of one cycle of the sinusoid sin(π/2) over −2≦t≦2. This sinusoid is an odd function. The even-indexed coefficients, C_{0,b} and C_{2,b} are zero since the inner product of an odd function and an even function is zero. If only two terms in the Hermite expansion Equation (6) are used, the first two nonzero coefficients in the discrete Hermite expansion correspond to u_{1,b} and u_{3,b}. The resulting two-term expansion with b=1 approximates this sinusoid with relative error averaging only 3.9%. Also, the first two non-zero coefficients in the expansion account for 99% of the coefficient energy (see FIG. 5).

One can also obtain the Fourier transform of the signal from the discrete Hermite expansion Equation (6). For the undilated case with b=1, the (centered) Fourier transform of u_{k,1} is simply j^{k}u_{k,1} for k=0,1, . . . ,n−1. This simple formula is once again similar to a property for the Fourier transform for continuous Hermite functions. Using this property and applying the centered Fourier transform to Equation (6), one finds that (for b=1)

$${F}_{C , 1} \cdot x = {\sum }_{k = 0}^{n - 1} ( {j}^{k} ) {c}_{k} {u}_{k} \qquad ( 8 )$$

so that one obtains the Fourier transform of the input signal without much extra computation. Although computation of u_{k,b} for b≧1 is very stable from T_{b}, this computation seems to become unstable if b<1.
Applications to ECG Signals

While not limited thereto, the present invention can be applied to ECGs in order to approximate and compress the ECG signals. In one embodiment, the method of the present invention is appropriate for the QRS complex of an ECG signal. The R pulse of the complex is a dominant feature, and methods have already been established to detect this complex within the ECG signal. If one of these methods is employed, the QRS complex may he centered with the maximum point of the R segment at the origin of an interval. The resulting QRS complex has the general shape similar to some of the low-indexed Hermite functions, such as u₂ and u₄ in FIG. 3. This suggests that a good approximation of Equation (6) for the QRS complex of the ECG signal may he accomplished using relatively few Hermite functions.

One of the difficulties in using continuous Hermite functions to approximate the QRS complex of an ECG signal is that a modem recording is both digital and finite in length, whereas the Hermite functions are continuous and are defined for all values of t. If the Hermite functions are simply sampled and the resulting vectors used for an expansion, those vectors are not orthogonal. Coefficients in such an expansion cannot he found by simple inner products as in Equation (7). However, the discrete dilated Hermite functions have the advantage in representing digital signals that they are an orthonormal set of signals where the expansion of the signal may he found easily and efficiently.

An advantage of this method in representing the QRS complex of an ECG signal is that the discrete dilated Hermite functions are localized. The u_{k,b} are concentrated near the origin for small k indices, and expand outward with greater width as the index k increases. In particular, if one considers u_{k,b}² as a probability distribution, then it can be shown that the standard deviation is approximately \sqrt{(2k=1)/(4π)}b, which increases with index k. If the signal to he modeled is concentrated near the origin, this property of Hermite functions makes it so that just a few of the first Hermite functions in the expansion shown in Equation (6) can give an excellent approximation.

For applications to ECG signals, the first set of examples assume that the QRS complex is about 200 ms in duration (which is conservative) and that 100 ms of zero values are added on the right and the left in order to center and isolate the QRS complex. Signals used here for the examples, as given in the following figures, were obtained from the following database—E. Traasdahl's ECG database as sponsored by the Signal Processing Information Base (SPIB), (see http://spib.rice.edu/spib/data/signals/medical/ecg_man.html). The database assumed a sampling rate of 1 kHz, so that signals used here have length n=400 samples: 200 samples of QRS complex data, and 100 zero samples at the beginning and end. The data were high-pass filtered to remove the dc component.

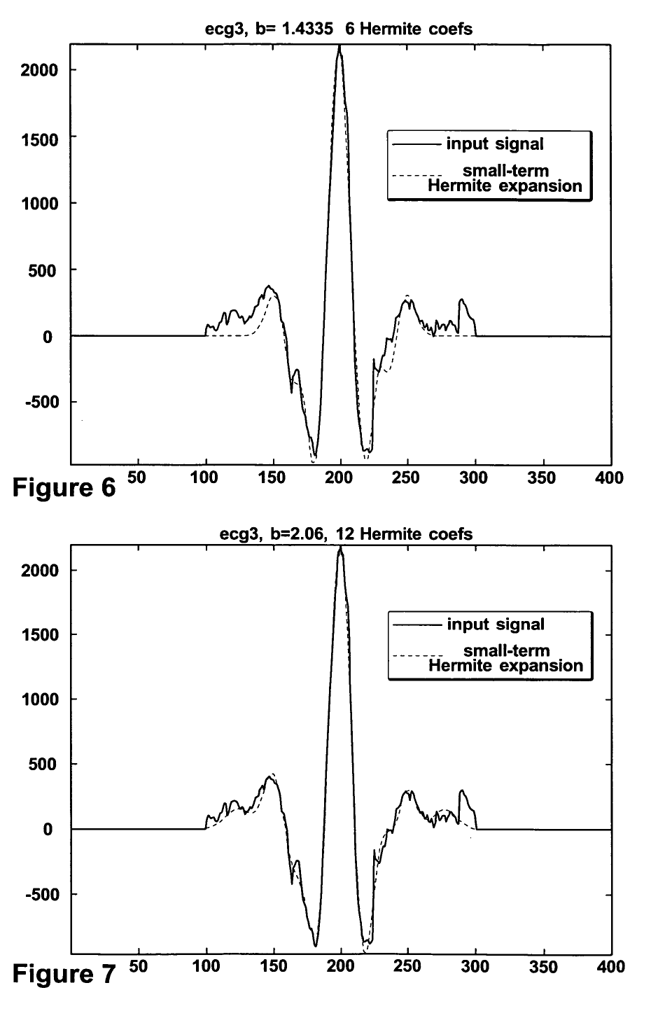

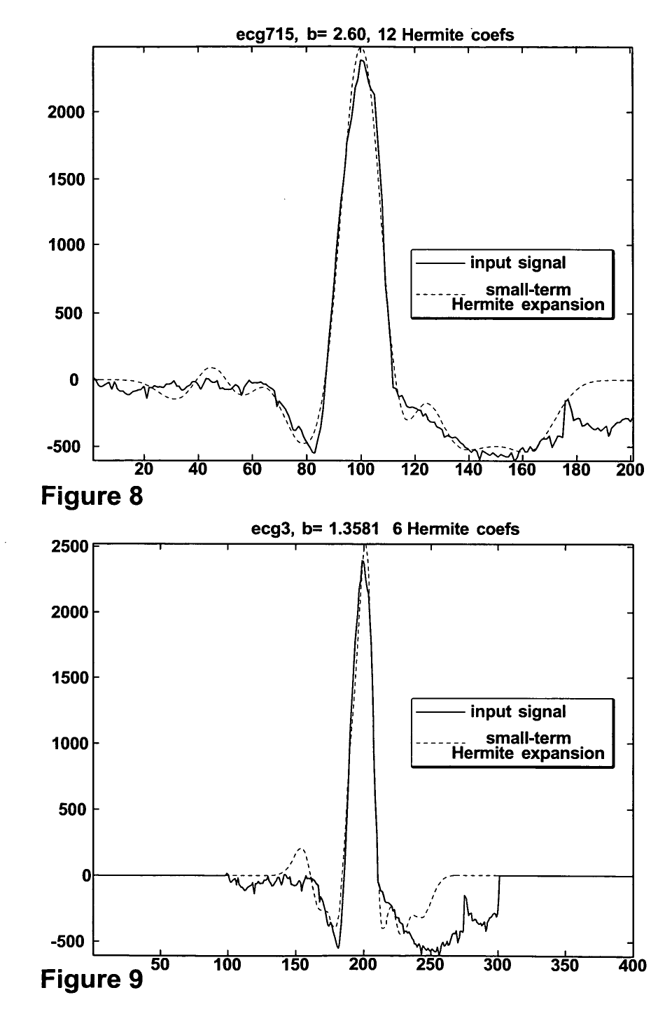

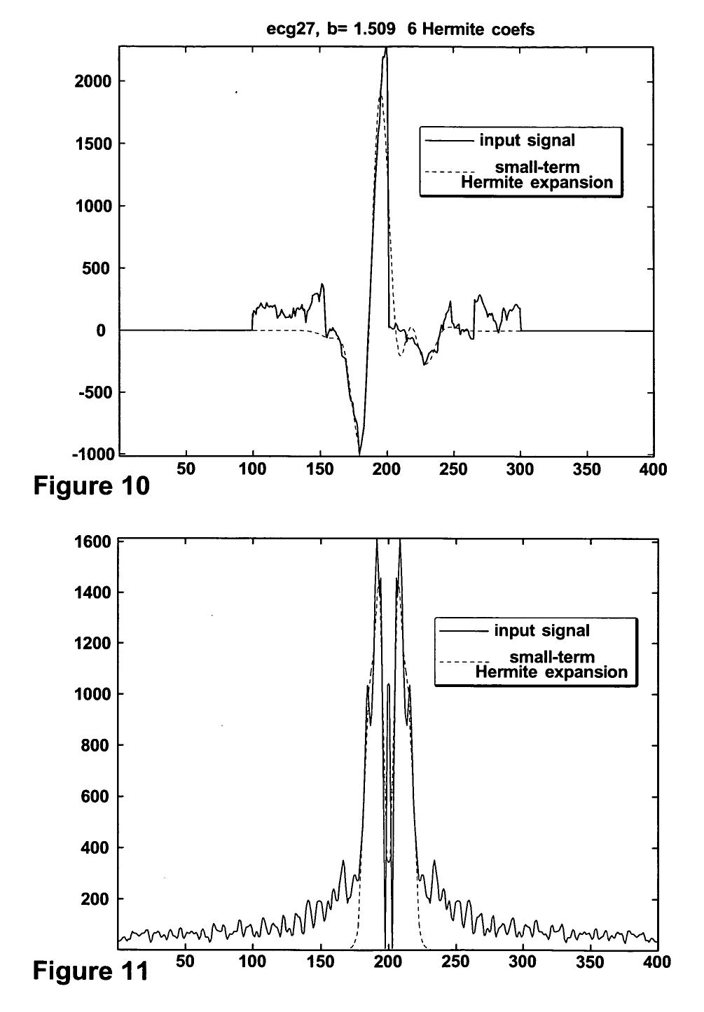

An expansion of a signal x in terms of discrete dilated Hermite functions as in Equation (6) includes the choice of the dilation parameter value b. Since the methods of the present invention involve fast computations, the choice of b is also based on a quick computation. As noted earlier, the general shape of the QRS complex is similar to u₂, although u₂ is symmetric and the QRS complex is generally not symmetric. If the positive-valued bumps outside of the QRS complex are included, then the shape is often similar to u₄. The criterion for choosing b that is favored, in one embodiment, by the present invention is based on u₂. In this embodiment, the minimum value of u₂ is matched with the minimum of the signal (Q or S) that is closer to the origin. Since u₂ is actually a vector, the choice of b is based on a discrete analysis instead of a continuous one. The actual equation used is shown below in Equation (9).

$$b = \frac{Δ t \cdot min ( xleft , xright )}{\sqrt{5 / 2} + 0.08} \qquad ( 9 )$$

where ‘xleft’ and ‘xright’ are the integer-valued horizontal distances from the origin to the input signal's minimum to the left and right of the origin, respectively. With this choice of dilation parameter b, signals such as those in FIGS. 6 and 10 are well-approximated using only six Hermite functions in the expansion of Equation (6). A formula similar to Equation (9) for b applies for when the match is for u₄, and results for those cases are shown in FIGS. 7 and 8, where a very good approximation of the signal is obtained with 12 Hermite functions. Compare FIG. 9 with six Hermite functions to FIG. 8 with 12. If the howl-shaped S portion of this signal is important for medical evaluations, then the approximation with 12 Hermite functions would he necessary. Finally, FIG. 11 shows that the (centered) Fourier transform of the discrete Hermite approximation has basically filtered the noisy transform of the ECG signal.
Further Expansion of ECG Signals

Expansion of ECG signals were conducted using the discrete Hermite expansion of signals detailed below. That is, given a digital signal x of length n, the discrete Hermite expansion (Equation (6)) of x is simply an expansion of an n-dimensional digital signal in a particular orthonormal basis. This expansion has the form shown in Equation (6) below.

$$x = {\sum }_{k = 0}^{n - 1} {c}_{k , b} {u}_{k , b} \qquad ( 6 )$$

with coefficients
c_{k,b}=<x,u_{k,b}> (7)
given by standard inner products of the input signal with the discrete dilated Hermite functions.

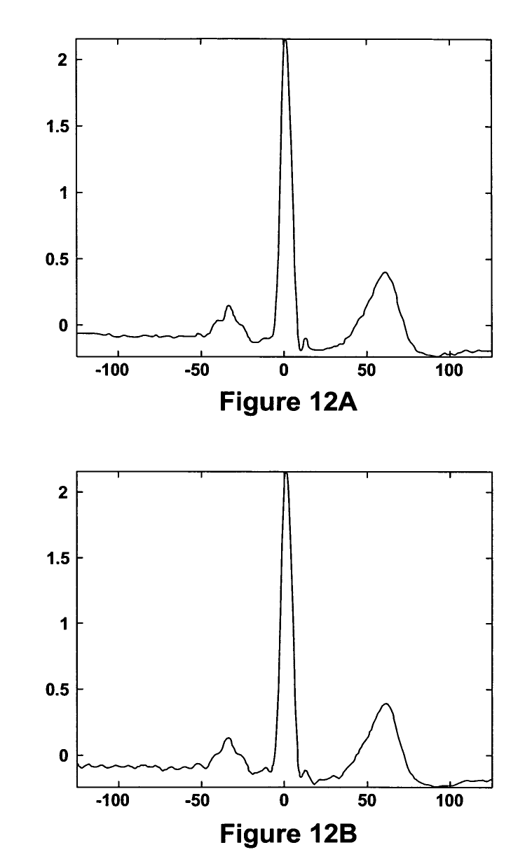

In light of the above, individual ECG complexes were centered at their R-peaks and the corresponding Hermite coefficients were calculated, using the principles outlined above. A dilation parameter of b=1 was used. The performance of the calculated Hermite coefficients in representing the ECG was calculated using the Percentage RMS Difference (PRD) error, given by

$$PRD = \sqrt{\frac{{\sum }_{i} {( {x}_{i} - {y}_{i} )}^{2}}{{\sum }_{i} {( {x}_{i} - \overline{x} )}^{2}}} \qquad ( 10 )$$

where xᵢ is the original ECG signal, yᵢ is the Hermite representation and \overline{x} is the mean of the signal. In this embodiment, the first 50 Hermite coefficients are sufficient for reconstructing the ECG with an acceptable PRD, although the present invention is not limited to just this embodiment. FIG. 12 shows a comparison of an original ECG signal and its reconstruction using the first 50 Hermite coefficients.

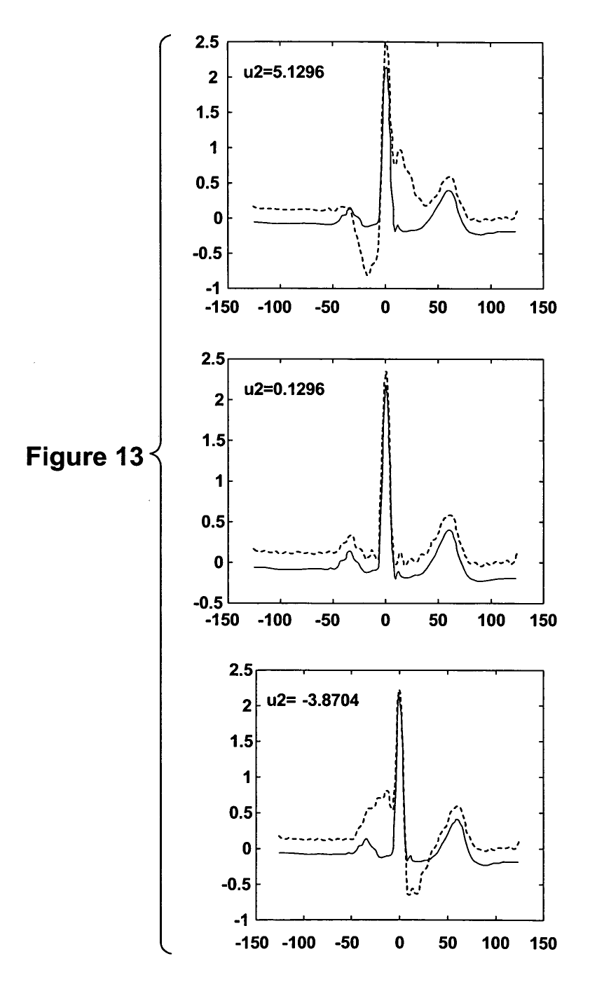

Changes in ECG features are reflected as variations in the values of the Hermite coefficients. As an example, FIG. 13 illustrates the contribution of the second Hermite function towards the reconstruction process, for coefficient values 5.1296, 0.1296 and −3.8704. As can be seen from FIG. 13, the second Hermite function with a large positive coefficient value fits ischemic features like deep Q wave and an ST segment elevation in the ECG. As the value approaches near zero, it fits a normal ECG. For a large negative value, it fits an ischemic ST depression feature. All of the 50 coefficient values, considered together, are a measure of the shape of the ECG and can be used as a tool for identification of ischemic features. However, in the absence of a clearly identifiable relationship between the coefficients and specific ECG features, a Neural Network based method was adopted.

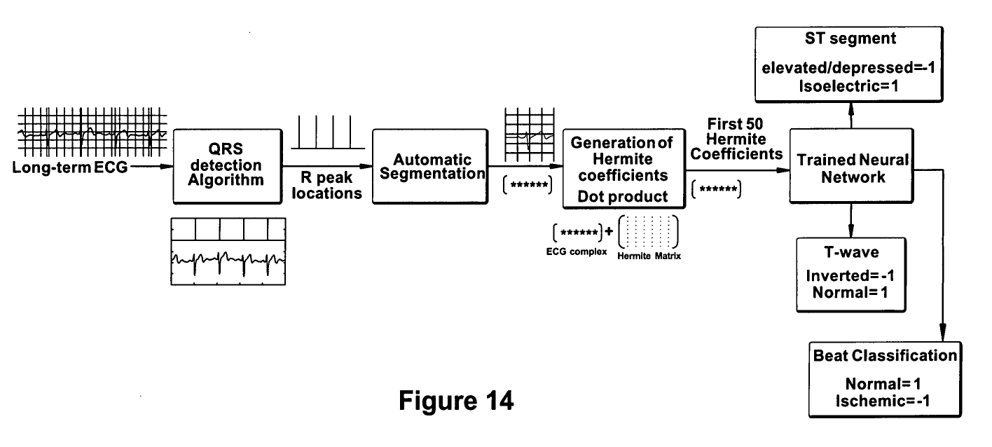

For long term ECG monitoring applications, an automated method for segmentation, Hermite expansion followed by classification was developed. One such scheme of the present invention is outlined in FIG. 14. Using a QRS detection algorithm, long term ECG signals were scanned for R-peak locations. This was used to automatically segment the ECG, with each ECG complex centered at its R-peak, and having a window size equivalent to the corresponding R-R interval. The Hermite coefficients corresponding to the individual ECG complexes were simultaneously calculated by a simple dot product as shown in Equation (7).

The first 50 coefficients were the input to a trained Neural Network classifier. The network outputs were the presence or absence of ST segment changes, T wave changes and ischemia.

Five Neural Networks were trained with the 50 Hermite coefficients as inputs. The networks had three layers with different number of hidden layer neurons. The 2 outputs of the network were presence/absence of ST segment changes and presence/absence of T-wave inversion. A committee of Neural Networks was used, since individual network results might vary in borderline ischemic cases. The majority decision of the committee of trained neural networks was used in arriving at the final classification.

Preliminary Training of a Committee of Neural Networks: The training data set consisted of 236 ECG complexes, containing both ischemic as well as normal ECG signals. The ECG signals were taken from the MIT-BIH database, predominantly from European ST-T database and long term ST-T database. The ischemic ECG signals were chosen based on 2 features viz. an elevated/depressed ST segment and an inverted T wave. All possible combinations of these two features were presented to the network. MATLAB Neural Network Toolbox was used for the training. The Conjugate gradient back propagation algorithm was used to train the Neural Networks.

Adaptive Training: In addition to the training, the network was retrained with a few samples of normal ECG cycle from each long term record that was used for testing. This was performed to show the network a .feel. of the normal ST segment and T-wave features from the particular long term ECG and to find out if the network was able to detect any changes in the ST and T wave features that occurred during ischemic episodes.

Testing: Twenty-four long-term ECG records from the European ST-T database were used to test the validity of the above method of the present invention, in simulated real-time conditions. The ECG records were continuously scanned for R-peak locations, by the methods described previously, and sets of 50 Hermite coefficients were simultaneously generated. The trained Neural Networks were used for beat-to-beat classification of the ECG, vis-à-vis ST segment and T wave changes (FIG. 14). A majority decision of the Committee of Neural Networks was used for arriving at the final decision.

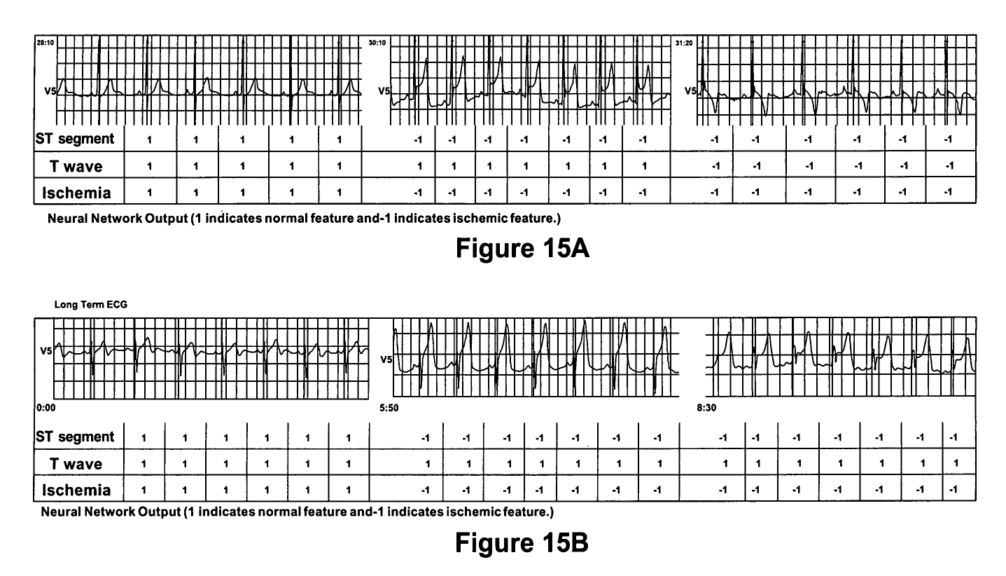

Results: A total of 1918 beats were used to test the trained networks. The results are tabulated in Table 1. For ST segment changes, a sensitivity of 97.2% and a specificity of 98.6% were observed. For T-wave inversion, a sensitivity of 98.6% and a specificity of 93.3% were observed. Overall, for ischemic episode detection, a sensitivity of 98% and a specificity of 97.3% were observed (Table 2). FIG. 15 shows the output of the committee of Neural Networks, in classifying a long term ECG record on a beat to beat basis.

TABLE 1
Results for Beat Classification

| | Ischemic Episode in ECG (Number of samples) | | | |
|---|---|---|---|---|
| | ST Segment Change | | T Wave Change | |
| Test Result | Present | Absent | Present | Absent |
| Positive | 1166 | 10 | 1179 | 48 |
| Negative | 33 | 709 | 20 | 671 |
| Total | 1199 | 719 | 1199 | 719 |

ST: Sensitivity = 1166/1199 (97.2%) and Specificity = 709/719 (98.6%)
T: Sensitivity = 709/719 (98.6%) and Specificity = 671/719 (93.3%)

TABLE 2
Results for Episode Classification

Ischemia (Number of samples)

| Test Result | Present | Absent |
|---|---|---|
| Positive | 1175 | 19 |
| Negative | 24 | 700 |
| Total | 1199 | 719 |

Sensitivity = 1175/1199 (98%) and Specificity = 700/719 (97.3%)

Comparison with Other Methods: Table 3 shows a comparison of sensitivity and specificity of some commonly used ischemia detection methods with the Hermite Function based approach. As can be seen from Table 3, the method of the present invention has comparable sensitivity and specificity in detecting ischemic episodes.

TABLE 3
Comparison Chart

| System Category | Sensitivity | Specificity |
|---|---|---|
| Digital Signal Analysis [1], [2] | 85.20 | — |
| Digital Signal Analysis [3] | 95.80 | 90.00 |
| Digital Signal Analysis [4] | 95.00 | 100.00 |
| Rule Based (ST Episodes) [5] | 92.02 | — |
| Rule Based (T Episodes) [5] | 91.09 | — |
| Fuzzy Logic [6] | 81.00 | — |
| Artificial Neural Networks [7] | 79.32 | 75.19 |
| Artificial Neural Networks [8] | 89.62 | 89.65 |
| Present Invention's Method | 98.00 | 97.30 |

[1] Technique based on Jager F, Mark R. G, Moody G. B, et al, “Analysis of Transient ST Segment Changes during Ambulatory ECG Monitoring using Karhunen-Loeve Transform”, Proc. IEEE Comput. Cardiol., pp. 691-694, 1992.
[2] Technique based on Jager F, Moody G, Mark R, “Detection of Transient ST Segment Episodes during Ambulatory ECG Monitoring”, Comput. Biomed Res., No. 31, pp. 305-322, 1998.
[3] Technique based on Baldilini F, Merri M, Benhorin J, et al. “Beat to Beat Quantification and Analysis of ST Ddisplacement from Holter ECGs: A New Approach to Ischemia Detection”, Proc. IEEE Comput. Cardiol., pp. 179-182, 1992.
[4] Technique based on Senhadji L, Carrault G, Bellanger J, et al. “Comparing Wavelet Transform for Recognizing Cardiac Pattern”, IEEE Eng. Med. Biol., No. 14(2): pp. 167-173, 1995.
[5] Technique based on C. Papaloukas, D. I. Fotiadis, A. Likas, et al. “Use of a Novel Rule Based Expert System in the Detection of Changes in the ST Segment and T Wave in Long Duration ECGs”, J. Electrocardiol., No. 35(1), pp. 105-112, 2001.
[6] Technique based on Vila J, Presedo J, Delgado M, et al. “SUTIL: Intelligent Ischemia Monitoring System”, Int J. Med. Inf., No. 47(3), pp. 193-214, 1997.
[7] Technique based on Stamkopoulos T, Diamantaras K, Maglaveras N, et al. “CG Analysis Using Nonlinear PCA Neural Networks for Ischemia Beat Detection,” IEEE Trans. Signal. Process, No. 46(11), pp. 3058-3067, 1998.
[8] Technique based on Maglaveras N, Stamkopoulos T, Diamantaras K, et al. “ECG Pattern Recognition and Classification using Linear Transformations and Neural Networks: A Review”, Int. J. Med. Inf., No. 52, pp. 191-208, 1998.

As mentioned above, the present invention is, in one embodiment, directed to a method for the real-time, automated identification of ischemic features from ECG signals. The method of the present invention is very effective in extracting shape features from the ECG signals. The computation of coefficients is simple and fast. The method of the present invention can be implemented for continuous bed side monitoring and offline inspection of ECG in ischemic patients. The results stated herein show an excellent sensitivity, which is crucial in bedside monitoring and screening of long term records. As discussed above, the present invention is not only limited to detecting/monitoring ischemia and/or ischemia-related abnormalities in ECGs. Rather, the present invention can be applied to a wide variety of ECG abnormalities and therefore used to track/diagnosis a variety of abnormalities in ECGs.

Although the invention has been described in detail with particular reference to certain embodiments detailed herein, other embodiments can achieve the same results. Variations and modifications of the present invention will be obvious to those skilled in the art and the present invention is intended to cover in the appended claims all such modifications and equivalents.

1. A method for monitoring/detecting abnormalities in an ECG, the method comprising the steps of:
(a) gathering at least one ECG;
(b) subjecting the at least one ECG to a QRS detection algorithm in order to scan for R-peak location;
(c) calculating the Hermite coefficients corresponding to the individual ECG complexes from each individual ECG; and
(d) subjecting the Hermite coefficients to a Neural Network in order to determine the present and/or absence of ECG abnormalities.

2. The method of claim 2, wherein Steps (b) and (c) are conducted simultaneously.

3. The method of claim 1, wherein the Hermite coefficients of Step (c) are calculated by a simple dot product.

4. The method of claim 1, wherein the ECG abnormalities being monitored are associated with cardiac ischemia.

5. The method of claim 4, wherein the Neural Network outputs the presence or absence of ST segment changes, T wave changes, beat classification, ischemia, or a combination of one or more thereof.

6. A computer system designed to carry out the method of claim 1, the computer system comprising:
at least one power source;
at least one input device;
at least one display; and
at least one memory device,
wherein the computer system is designed to act as a Neural Network.

7. A method for monitoring/detecting abnormalities in an ECG, the method comprising the steps of:
(a) gathering at least one ECG;
(b) subjecting the at least one ECG to a QRS detection algorithm in order to scan for R-peak location;
(c) calculating the Hermite coefficients corresponding to the individual ECG complexes from each individual ECG; and
(d) subjecting the Hermite coefficients to a Neural Network in order to determine the present and/or absence of ECG abnormalities,
wherein the ECG abnormalities being monitored/detected are associated with cardiac ischemia, and wherein Steps (b) and (c) are conducted simultaneously.

8. The method of claim 7, wherein the Hermite coefficients of Step (c) are calculated by a simple dot product.

9. The method of claim 7, wherein the Neural Network outputs the presence or absence of ST segment changes, T wave changes, beat classification, ischemia, or a combination of one or more thereof.

10. A computer system designed to carry out the method of claim 7, the computer system comprising:
at least one power source;
at least one input device;
at least one display; and
at least one memory device,
wherein the computer system is designed to act as a Neural Network.
